SEQUENCE LISTING (48 sequences)

SEQ ID NO: 1 - DNA, 19 bp, Artificial Sequence
  Oligonucleotide Primer
atggatgacgatatcgctg

SEQ ID NO: 2 - DNA, 19 bp, Artificial Sequence
  Oligonucleotide Primer
atgaggtagtctgtcaggt

SEQ ID NO: 3 - DNA, 20 bp, Artificial Sequence
  Oligonucleotide Primer
ggagtgtcgcttagaggtgc

SEQ ID NO: 4 - DNA, 20 bp, Artificial Sequence
  Oligonucleotide Primer
tccagaaagccaagagaagc

SEQ ID NO: 5 - DNA, 22 bp, Artificial Sequence
  Oligonucleotide Primer
tagtgaccagctataatcagag

SEQ ID NO: 6 - DNA, 20 bp, Artificial Sequence
  Oligonucleotide Primer
acgccaaggtctgaaggtcc

SEQ ID NO: 7 - DNA, 19 bp, Artificial Sequence
  Oligonucleotide Primer
ccctgctggccctgctctt

SEQ ID NO: 8 - DNA, 20 bp, Artificial Sequence
  Oligonucleotide Primer
aggtctgaaggtcacctgct

SEQ ID NO: 9 - DNA, 19 bp, Artificial Sequence
  Oligonucleotide Primer
tcatgacgtttggcaagtt

SEQ ID NO: 10 - DNA, 20 bp, Artificial Sequence
  Oligonucleotide Primer
cagaggagaaccccagatca

SEQ ID NO: 11 - DNA, 20 bp, Artificial Sequence
  Oligonucleotide Primer
gattccctatttggatcccc

SEQ ID NO: 12 - DNA, 20 bp, Artificial Sequence
  Oligonucleotide Primer
ctctctgtggcactgaacca

SEQ ID NO: 13 - DNA, 19 bp, Artificial Sequence
  Oligonucleotide Primer
ccacccagtttacaagctc

SEQ ID NO: 14 - DNA, 20 bp, Artificial Sequence
  Oligonucleotide Primer
tgtaggcagtacgggtcctc

SEQ ID NO: 15 - DNA, 20 bp, Artificial Sequence
  Oligonucleotide Primer
tgtaggcagtacgggtcctc

SEQ ID NO: 16 - DNA, 20 bp, Artificial Sequence
  Oligonucleotide Primer
ccaccccagtttacaagctc

SEQ ID NO: 17 - DNA, 20 bp, Artificial Sequence
  Oligonucleotide Primer
cattgttgcaccttgtcacc

SEQ ID NO: 18 - DNA, 20 bp, Artificial Sequence
  Oligonucleotide Primer
ttctgctgctttccctcatt

SEQ ID NO: 19 - DNA, 20 bp, Artificial Sequence
  Oligonucleotide Primer
gcaaatgtgtgtttgatgcc

SEQ ID NO: 20 - DNA, 20 bp, Artificial Sequence
  Oligonucleotide Primer
atgaccaaactcttggaccg

SEQ ID NO: 21 - DNA, 18 bp, Artificial Sequence
  Oligonucleotide Primer
cgccgcctgtccgcttcc

SEQ ID NO: 22 - DNA, 24 bp, Artificial Sequence
  Oligonucleotide Primer
ttgggcttccgttttctggtttga

SEQ ID NO: 23 - DNA, 20 bp, Artificial Sequence
  Oligonucleotide Primer
acctgagtccgagtctgacc

SEQ ID NO: 24 - DNA, 20 bp, Artificial Sequence
  Oligonucleotide Primer
ggcaccttgagaaagcagtc

SEQ ID NO: 25 - DNA, 24 bp, Artificial Sequence
  Oligonucleotide Primer
ggcgttctctttggaaaggtgttc

SEQ ID NO: 26 - DNA, 20 bp, Artificial Sequence
  Oligonucleotide Primer
ctcgaaccacatccttctct

SEQ ID NO: 27 - DNA, 23 bp, Artificial Sequence
  Oligonucleotide Primer
tgaagagagcggagaaggagatc

SEQ ID NO: 28 - DNA, 24 bp, Artificial Sequence
  Oligonucleotide Primer
tctggagttaagaaatcggagctg

SEQ ID NO: 29 - DNA, 20 bp, Artificial Sequence
  Oligonucleotide Primer
acctgttgacggattccaag

SEQ ID NO: 30 - DNA, 20 bp, Artificial Sequence
  Oligonucleotide Primer
tcatgaggaagcgtaggtcc

SEQ ID NO: 31 - DNA, 24 bp, Artificial Sequence
  Oligonucleotide Primer
tcaagactgactcacagcaacccc

SEQ ID NO: 32 - DNA, 24 bp, Artificial Sequence
  Oligonucleotide Primer
ctttgtcctgaaccgtggtggtag

SEQ ID NO: 33 - DNA, 20 bp, Artificial Sequence
  Oligonucleotide Primer
cctcctttacggtggacaaa

SEQ ID NO: 34 - DNA, 20 bp, Artificial Sequence
  Oligonucleotide Primer
atcaactcctcctgccaatg

SEQ ID NO: 35 - DNA, 24 bp, Artificial Sequence
  Oligonucleotide Primer
ggaagatcacaagaaactccgaac

SEQ ID NO: 36 - DNA, 23 bp, Artificial Sequence
  Oligonucleotide Primer
ggatgcgagctttggattcatag

SEQ ID NO: 37 - DNA, 22 bp, Artificial Sequence
  Oligonucleotide Primer
gctgttcgcaaagactcgctac

SEQ ID NO: 38 - DNA, 25 bp, Artificial Sequence
  Oligonucleotide Primer
ccatgacctatactcaggcttcagg

SEQ ID NO: 39 - DNA, 25 bp, Artificial Sequence
  Oligonucleotide Primer
ccatgacctatactcaggcttcagg

SEQ ID NO: 40 - DNA, 26 bp, Artificial Sequence
  Oligonucleotide Primer
gaagctccatatccctgggtggaaag

SEQ ID NO: 41 - DNA, 23 bp, Artificial Sequence
  Oligonucleotide Primer
ccaaagtggtggacaagattgcc

SEQ ID NO: 42 - DNA, 24 bp, Artificial Sequence
  Oligonucleotide Primer
gggataggaaggacgctcaaagac

SEQ ID NO: 43 - DNA, 24 bp, Artificial Sequence
  Oligonucleotide Primer
cagatgtagtccgccaaaggatag

SEQ ID NO: 44 - DNA, 26 bp, Artificial Sequence
  Oligonucleotide Primer
atgccactgatggagtatgaggagcc

SEQ ID NO: 45 - DNA, 20 bp, Artificial Sequence
  Oligonucleotide Primer
ctgagagtgccagaaaaggg

SEQ ID NO: 46 - DNA, 20 bp, Artificial Sequence
  Oligonucleotide Primer
tcatcatgctggagaactcg

SEQ ID NO: 47 - DNA, 22 bp, Artificial Sequence
  Oligonucleotide Primer
atcctcgggagatgacgaagac

SEQ ID NO: 48 - DNA, 23 bp, Artificial Sequence
  Oligonucleotide Primer
ggatgctgccaaactttgttctc

(19) United States
(12) Patent Application Publication
(10) Pub. No.: US 2004/0072344 A1
(43) Pub. Date: Apr. 15, 2004

(54) Method for inducing differentiation of embryonic stem cells into functioning cells

(76) Inventors: Kazutomo Inoue, Kyoto-shi (JP); Dohoon Kim, Kyoto-shi (JP); Yanjun Gu, Kyoto-shi (JP); Michiyo Ishii, Kyoto-shi (JP)

Correspondence Address: BIRCH STEWART KOLASCH & BIRCH, PO BOX 747, FALLS CHURCH, VA, 22040-0747, US

(21) Appl. No.: 10/626,772

(22) Filed: Jul. 25, 2003

Related U.S. Application Data
(63) Continuation-in-part of application No. 10/054,789, filed on Jan. 25, 2002.

(51) Int. Cl. C12N 5/08

(52) U.S. Cl. 435/366

(57) ABSTRACT

The present invention provides a 4-step method for inducing differentiation of embryonic stem cells into functioning cells comprising 1) expanding ES cells; 2) inducing Embryoid Bodies in the presence of leukemia inhibitory factor and basic FGF; 3) selection expanding of the EBs and 4) differentiation. According to the present invention, ES cells can be differentiated into either insulin producing pancreatic islet like cell clusters or nerve like cells. Thus obtained functioning cells may be potential sources of donor cells in cell transplant therapy for many patients.

### CROSS-REFERENCE TO RELATED APPLICATIONS

This application is a continuation-in-part of U.S. patent application Ser. No. 10/054,789 filed Jan. 25, 2002.

### BACKGROUND OF THE INVENTION

1. Field of the Invention

The present invention relates to a method for inducing differentiation of mammalian embryonic stem cells into functioning cells. The present invention also relates to the functioning cells obtained by the present invention and a method for treatment of a patient by implanting or introducing the functioning cells to the patient.

2. Art Related

Pluripotent stem cells have been derived from two embryonic sources. Embryonic stem (ES) cells are derived from the inner cell mass of preimplantation embryos, and embryonic germ (EG) cells are derived from primordial germ cells (PGCs). Both ES and EG cells are pluripotent and demonstrate germ-line transmission in experimentally produced chimeras. Mouse ES and EG cells share several morphological characteristics such as high levels of intracellular alkaline phosphatase (AP), and presentation of specific cell surface glycolipids and glycoproteins. These properties are characteristic of, but not specific for, pluripotent stem cells. Other important characteristics include growth as multicellular colonies, normal and stable karyotypes, the ability to be continuously passaged, and the capability to differentiate into cells derived from all three embryonic germ layers. Pluripotent stem cell lines that share most of these characteristics also have been reported for chicken, mink, hamster, pig, rhesus monkey, and common marmoset. Also a stem cell is a cell that has the ability to divide (self-replication) for indefinite periods—often throughout the life of the organism. Under the right conditions, or given the right signals, stem cells can give rise (differentiate) to the many different cell types that make up the organism.

Recently, S. H. Lee et al. (Nature Biotechnology 18, 675-679 (2000), the disclosure of the publication is herein incorporated by reference) disclosed to generate CNS progenitor populations from ES cells, to expand these cells and to promote their differentiation into dopaminergic and serotonergic neurons in the presence of mitogens and specific signaling molecules. The differentiation and maturation of neuronal cells was completed after mitogen withdrawal from the growth medium. This experimental system provides a powerful tool for analyzing the molecular mechanisms controlling the functions of these neurons in vitro and in vivo, and potentially for understanding and treating neurodegenerative and psychiatric diseases.

Also, H. Kawasaki et al. (Neuron 28, 31-40(2000), the disclosure of the publication is herein incorporated by reference) have identified a stromal cell-derived inducing activity (SDIA) that promotes neural differentiation of mouse ES cells. SDIA accumulates on the surface of PA6 stromal cells and induces efficient neuronal differentiation of co-cultured ES cells in serum-free conditions without use of either retinoic acid or embryonic bodies. BMP4, which acts as an antineuralizing morphogen in Xenopus, suppresses SDIA-induced neuralization and promotes epidermal differentiation. A high proportion of tyrosine hydroxylase-positive neurons producing dopamine are obtained from SDIA-treated ES cells. When transplanted, SDIA-induced dopaminergic neurons integrate into the mouse striatum and remain positive for tyrosine hydroxylase expression. Neural induction by SDIA provides a new powerful tool for both basic neuroscience research and therapeutic applications.

In a study of B. Soria et al., mouse embryonic stem cells have been introduced as a new potential source for cell therapy in type 1 diabetic patients (Diabetes 49: 157-162 (2000), the disclosure of the publication is herein incorporated by reference). Using a cell-trapping system, they have obtained an insulin-secreting cell clone from undifferentiated ES cells. The construction used allows the expression of a neomycin selection system under the control of the regulatory regions of the human insulin gene. The chimeric gene also contained a hygromycin resistance gene (pGK-hygro) to select transfected cells. A resulting clone (IB/3×-99) containing 16.5 ng/μg protein of total insulin displays regulated hormone secretion in vitro in the presence of various secretagogues. Clusters obtained from this clone were implanted in the spleen of streptozotocin-induced diabetic animals. Hyperglycemia of the transplanted animals were normalized within one week and their body weight were restored in 4 weeks. Whereas slower recovery was observed in the transplanted animals than control mice in an intraperitoneal glucose tolerance test, blood glucose levers after meal load were normalized in a similar manner. This approach opens new possibilities for tissue transplantation in the treatment of type 1 and type 2 diabetes and offers an alternative to gene therapy.

S. Assady et al. (Diabetes 50: 1691-1697 (2001), the disclosure of the publication is herein incorporated by reference), used pluripotent undifferentiated human embryonic stem cells (hES) as a model system for lineage-specific differentiation. They cultured hES cells in both adherent and suspension culture conditions, and observed spontaneous in vitro differentiation of the cells including generation of cells with characteristics of insulin-producing β-cells. Immunohistochemical staining for insulin was observed in a surprisingly high percentage of the cells. Secretion of insulin into the medium was observed in a differentiation-dependent manner and was associated with the appearance of other β-cell markers. These findings suggest that the hES cell model system is a potential basis for enrichment of human β-cells or their precursors, as a possible future source for cell replacement therapy in diabetes.

Su-Chun Zhang et al. (Nature Biotech. 19, 1129-1133 (2001), the disclosure of the publication is herein incorporated by reference) disclose in vitro differentiation, enrichment, and transplantation of neural precursor cells from human ES cells. Upon aggregation to embryoid bodies, differentiating ES cells formed large numbers of neural tube-like structures in the presence of fibroblast growth factor 2 (FGF-2). Neural precursors within these formations were isolated by selective enzymatic digestion and further purified on the basis of differential adhesion. Following withdrawal of FGF-2, they differentiated into neurons, astrocytes, and oligodendrocytes. After transplantation into the neonatal mouse brain, human ES cell-derived neural precursors were incorporated into a variety of brain regions, where they differentiated into both neurons and astrocytes. No teratoma formation was observed in the transplant recipients. These results depict human ES cells as a source of transplantable neural precursors for possible nervous system repair.

Nadya Lumelsky et al. disclose a series of experiments in which they induced mouse embryonic cells to differentiate into insulin-secreting structures that resembled pancreatic islet (Science 292, 1389-1394 (2001), the disclosure of the publication is incorporated herein by reference). They have generated cells expressing insulin and other pancreatic endocrine hormones from mouse ES cells. The cells self-assemble to form three-dimensional cluster similar in topology to normal pancreatic islets where pancreatic cell types are in close association with neurons. Glucose triggers insulin release from these cell clusters by mechanisms similar to those employed in vivo. When injected into diabetic mice, the insulin-producing cells undergo rapid vascularization and maintain a clustered, islet-like organization.

However, the insulin-producing cells obtained by Lumelsky did not express pancreatic specific markers, amylase and carboxypeptidase. Further, Lumelsky grafted the insulin-producing cells into a diabetic model animal but failed to observe a sustained correction of hyperglycemia in the model animal.

Seven million people in Japan and 16 million people in the United States are affected by type I diabetics. At present, daily insulin administration or allogenic pancreas transplantation is employed for treatment of diabetics. Although the overall success rates of the pancreas transplantation have significantly increased, organ transplantation requires very invasive surgery and life-long immunosuppressive treatments, which significantly strain the patient. Further, availability of donor organs is still a serious problem preventing the operation to be popular. Therefore, development of a simple and universal treatment for diabetes is desired.

### SUMMARY OF THE INVENTION

An object of the present invention is to provide a novel method for inducing differentiation of pluripotent embryonic stem cells into functioning cells, especially pancreatic islet like cell clusters or nerve like cells.

Another object of the present invention to provide a method for treating a patient having disorders in pancreatic islet function.

Further object of the present invention is to provide a method for treating a patient having neuronal degeneration or spinal code disorders.

Still further, another object of the present invention is to provide functioning cells which are derived from mammalian ES cells and exhibit pancreatic islet like or nerve like functions.

Accordingly, the present invention provides a method for inducing differentiation of mammalian embryonic stem cells into functioning cells, which comprises the steps of;

1) culturing the mammalian embryonic stem cells together with feeder cells with a medium comprising leukemia inhibitory factor;

2) culturing the obtained cells in absence of feeder cells with a medium comprising leukemia inhibitory factor and basic fibroblast growth factor (hereinafter referred to as “bFGF”) in suspension culture condition to give embryonic bodies;

3) culturing the obtained embryonic bodies with a growth-selection medium; and

4) culturing the obtained cell clusters with a differentiation medium to give functioning cells.

According to the present invention, functioning cells such as pancreatic islet like cell clusters or nerve like cells can be differentiated from the mammalian ES cells.

The pancreatic islet like cell clusters induced by the present invention have an ability to produce insulin and to secrete insulin in response to glucose stimulation. The cells form clusters expressing pancreatic-related endocrine and exocrine markers including insulin, glucagon, Glut-2, islet amyloid polypeptide, amylase and carboxypeptidase.

The nerve like cells induced by the present invention exhibit nerve fiber like appearance. The cells form clusters expressing nerve related markers including nestin, β-tublin III, serotonin and tyrosine hydroxylase Nurt 1.

The inventors grafted the insulin-secreting islet like cell clusters induced from mouse ES cells by the method of the present invention into streptozotocin induced diabetic mice, and succeeded in decreasing the high blood glucose levels of the diabetic mice to those around the normal level. This study supports that the insulin producing islet like cell clusters obtained by the invention are useful for treatment of diabetics.

The present invention further provides a method for treating a mammalian patient having disorders in pancreatic islet function, which comprises the step of transplanting islet-like cell clusters induced from allogenic ES cells according to the invention to the patient.

The present invention also provides a method for treating a patient with nerve degenerative disease or spinal cord injury, which comprises the step of transplanting nerve like cells induced from allogenic ES cells according to the present invention to the patient.

Further, the present invention also provides functioning cells including pancreatic islet like cell clusters and nerve like cells derived from the mammalian ES cells by the method of the present invention. The functioning cells are useful not only for cell transplant therapy but also for in vitro screening of various new drugs which affect or restore islet or nerve function, safety evaluation of new drugs and so on.

### BRIEF DESCRIPTION OF THE DRAWINGS

FIG. 1 is a schematic description of differentiation steps of the present invention from ES cells to functioning cells.

FIG. 2 represents the results of insulin secretion from the cell clusters obtained in Example 1 in response to glucose stimulation. In this graph, column L represents the amount of insulin secreted per cluster in response to low dose (3.3 mg/L) glucose stimulation determined 5 minutes and 30 minutes respectively after the stimulation. Column H represents the amount in response to high dose (25 mmol/L) glucose stimulation.

FIG. 3 represents time-course of non-fasting blood glucose levels of diabetic mice implanted with the pancreatic islet like cell clusters derived from mouse ES cells compared to that of a control group.

FIG. 4 represents time course of body weight of diabetic mice implanted with the pancreatic islet like cell clusters derived from ES cells compared to that of a control group.

FIG. 5 represents percentages of TuJ-positive cell colonies induced from mouse ES cells by culturing in the presence of 0(control), 5 and 10 mM nicotinamide in steps 3 and 4.

FIG. 6 represents percentages of TuJ-positive cell colonies induced from mouse ES cells by culturing in the presence or absence of 4 ng/ml of bFGF (treated or untreated respectively) and then, in the presence of 10 mM nicotinamide (steps 3 and 4).

FIG. 7 is a histogram summarizing the effect of bFGF on the proportion of EBs (in step 4) displaying different degrees of network complexity.

FIG. 8 represents effects of BMP-4 and FBS on expression of TuJ on the colonies induced from mouse ES cells.

FIGS. 9-11 represent effects of transplantation of the differentiated and undifferentiated ES cells on 6-OHDA lesioned rats.

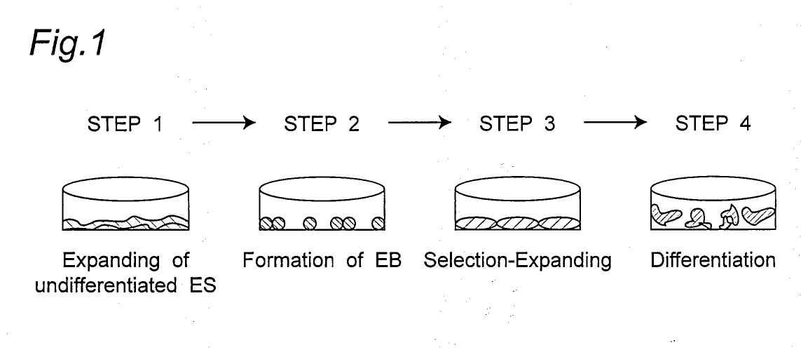

### DETAILED DESCRIPTION OF THE INVENTION

In the specification, claims and drawings of the instant application, the term “embryonic stem cell(s)” or “ES cell(s)” represents pluripotent cells derived from the inner cell mass of in vitro fertilized blastocytes.

Embryoid body or EB represents a cell cluster composed of three embryonic germ layers and formed from ES cells on their in vitro aggregation.

The feeder cell layer as used herein may be constructed in accordance with any procedure known in the art, and may be prepared from mice fatal fibroblast cells. Feeder cells are now commercially available.

The mammalian ES cells which may be used herein are not limited and may be rodent, such as mouse ES cells and rat ES cells, as well as primate such as cynomolgus ES cells and human ES cells. At present, various ES cells are derived and available including mice and human EC cells. Alternatively, the ES cells used herein may be those obtained from mammalian fertilized ovum by means of previous reports. For example, techniques for isolating stable cultures of human embryonic stem cells have been described by Thomson et al. (U.S. Pat. Nos. 5,843,780 and 6,200,806; Science vol. 282 1145-1147 (1998), the disclosure of these publications are herein incorporated by reference).

Step 1 of the present method is a conventional ES cell propagation step, which is described in, such as, N. Lumelsky et al., Science 292, 1389-1394 (2001), the disclosure of the publication is herein incorporated by reference.

Typically, mouse fetal feeder cells are cultured on a gelatin coated cell culture container to give a layer on the inner surface, then the ES cells are plated on the layer and cultured with an ES cell proliferating medium comprising leukemia inhibitory factor (hereinafter, referred to as “LIF”). By culturing under such condition as above, ES cells proliferate in an undifferentiated state.

In the method of the present invention, feeder cells may be those commercially available cells or those derived from mice fetal fibroblast cells by a conventional manner.

The ES cell proliferating medium used in step 1 may comprise 100-10000 U/ml of LIF. As a medium used in this step, any known medium that contains LIF and is useful for ES cell proliferation can be employed. An especially preferable medium is high glucose Dulbecco's modified Eagle's medium (Life Technology (herein below, Life Tech,), Grand, N.Y.) supplemented with 20% fetal bovine serum replacement (Life Tech.), 2% nonessential amino acid (Life Tech.), 0.1 mmol/l 2-mercaptoethanol (Life Tech.), 1000 U/ml of leukemia inhibitory factor (LIF; Life Tech.) and 2 mmol/1 L-glutamine (Life Tech.).

In step 1, culture of the ES cells may be continued until a desired amount of the cells is obtained. Typically, 3-7 days culture may provide enough cells. The obtained ES cells are transferred to the next step.

Throughout the inducing method of the present invention, culture of the cells or cell clusters may be carried out under a conventional cell culture condition such as at 37° C., in a humidified atmosphere of 5% CO₂ in 95% air.

In step 2, the proliferated ES cells are kept in suspension culture with a medium supplemented with LIF and bFGF. Heretofore, LIF has been believed to help retain the ES cells in an undifferentiated state and the art has believed that it is indispensable to exclude LIF from the culture in order to induce differentiation of the ES cells. Accordingly, as far as known to the inventors, all of the proposed EB inducing conditions contain the step culturing the expanded ES cells in suspension culture with a medium containing no LIF to allow their aggregation (for example, Su-Chen Zhang et al., Nature biotechnology, 19, 1129-1133 (2001), the disclosure of the publication is herein incorporated by reference).

The present inventors, however, succeeded to provide highly efficient EB formation from the ES cells with a medium comprising LIF and bFGF.

The medium used in step 2 contains LIF and bFGF. The amount of LIF in the medium may preferably be about 100-10000 U/ml. The amount of bFGF in the medium may preferably be about 2-100 ng/ml. The medium used in this step may further comprise one or more growth factors such as activin and nerve growth factor, cytokines such as interleukin-1 and interleukin-2, vitamins such as rethinol and nicotinamide, additional amino-acids such as tyrosine and lysine, and extra cellular matrixes such as fibronectin, laminin, collagen and heparin in a conventional chemically defined cell culture medium. An example of especially preferred medium used in this step is high glucose Dulbecco's modified Eagle's medium (Life Tech.) supplemented with 20% fetal bovine serum replacement (Life Tech.), 2% nonessential amino acid (Life Tech.), 0.1 mmol/l 2-mercaptoethanol (Life Tech.), 1000 U/ml of leukemia inhibitory factor (LIF; Life Tech.), 2 mmol/l of L-glutamine (Life Tech.) and 4 ng/ml of bFGF (R&D systems, Minneapolis).

In step 2, the ES cells are cultured in suspension without the feeder cell layer to allow the cells to aggregate to give embryoid bodies. The formation of EBs may be microscopically monitored. According to the present invention, EB formation may be observed from 2 days of the suspension culture. The suspension culture may be continued for 5-10 days to obtain enough amount of EBs. According to the present invention, a significantly larger number of vital EBs are induced than those induced by a conventional suspension culture step with a medium containing no LIF nor bFGF.

The EBs obtained in step 2 are then transferred to a selection-expansion step (step 3). In step 3, thus obtained EBs are plated on a culture container of which the inner surface is coated with a protein, such as collagen type IV, and cultured with an appropriate selection-expansion medium. It is preferable to culture the EBs in the protein coated container with the medium used in step 2 for about 2 days and then exchange the medium with a selection-expanding medium.

The selection-expanding medium used in step 3 may preferably be a serum-free cell culture medium supplemented with nicotinamide, insulin and fibronectine. The medium used in this step may further comprise one or more growth factors such as activin and nerve growth factor, cytokines such as interleukin-1 and interleukin-2, vitamins such as rethinol, additional amino-acids such as tyrosine and lysine, and extra cellular matrixes such as laminin, collagen and heparin in a conventional chemically defined cell culture medium. An example of preferable medium is a serum free DMEM/F-12 medium supplemented with nicotinamide, fibronectine, and N-2 supplements (GIBCO, 17502-014: consisting of Insulin 500 μg/ml, Human transferrin 10000 μg/ml, Progesterone 0.63 μg/ml, Putrescine 1611 μg/ml and Selenite 0.52 μg/ml in water).

In step 3, the EBs may be cultured with the selection-expanding medium for 3-14 days, preferably for 4-7 days.

The cell clusters obtained in step 3 are then dissociated from the container and plated on a culture container of which inner surface is coated with a protein or an amino acid. The transferred clusters are further cultured in a differentiation medium.

In step 4, the cell clusters can be differentiated into either pancreatic islet like cell clusters or nerve like cells.

In case the islet like cell clusters are desired, the cell clusters may be cultured with a serum-free cell culture medium supplemented with nicotinamide, insulin and laminin. The medium may further comprise one or more growth factors such as activin and nerve growth factor, cytokines such as interleukin-1 and interleukin-2, vitamins such as rethinol, additional amino-acids such as tyrosine and lysine, and extra cellular matrixes such as fibronectin, collagen and heparin in a conventional chemically defined cell culture medium. An especially preferred example is serum-free DMEM/F12 medium supplemented with nicotinamide, laminin and N-2 supplement.

According to the present invention, in order to differentiate into nerve like cells, the cell clusters may be cultured with a serum-free cell culture medium supplemented with lysine and laminin. The medium may further comprise one or more growth factors such as activin and nerve growth factor, cytokines such as interleukin-1 and interleukin-2, vitamins such as rethinol and nicotinamide, additional amino-acids such as tyrosine and lysine, and extra cellular matrixes such as fibronectin, collagen and heparin in a conventional chemically defined cell culture medium. An especially preferred example is serum-free DMEM/F12 medium supplemented with lysine, laminin and N-2 supplement.

In step 4, cell clusters may be cultured for 3-90 days or longer. 4-12 days culture will be enough for differentiation into the desired functioning cells and further culture may provide further proliferation of the differentiated clusters.

The pancreatic islet like cell clusters obtained by the present invention represent an ability to produce insulin and to secrete insulin in response to glucose stimulation, and the cells form clusters expressing genes specific to pancreatic endocrine cells including insulin, glucagon, Glut-2 and islet amyloid polypeptide as well as those specific to pancreatic exocrine cells including amylase and carboxypeptidase.

The nerve like cells obtained by the present invention represent nerve fiber like appearances and express markers relevant to nerve cells including nestin, b-tublin III, seletonin, tyrosine hydroxylase. Therefore, said nerve like cells are capable of generating mature neurons.

Since mice ES cells as well as human ES cells proliferate in vitro in an undifferentiated state retaining the pluripotency for more than one year, the present method can be employed to provide enough amounts of donor cells used in the cell transplanting therapy.

The present invention further provides a method for treating a mammalian patient having disorders in pancreatic islet function, which comprises transplanting islet-like cell clusters induced from allogenic ES cells according to the invention to the patient. In the present invention, “mammalian patient having disorders in pancreatic islet function” includes, but not limited to, type I diabetic patient, pancreatomized patient and insulin-dependent diabetic patient such as type II diabetic patient or patient with cystic fibrosis. The mammalian patient may include a human patient.

In this embodiment, transplantation of the pancreatic islet like cell clusters obtained as above may be carried out according to a clinically performed or proposed islet transplantation protocol (for example, Kazutomo Inoue and Masaaki Miyamoto, J. Hepatobiliary Pancreat. Surg. 7: 163-177 (2000), and Wenjing Wang et al., Transplantation 73: 122-129 (2002); the disclosure of the publications are herein incorporated by reference). For example, the pancreatic islet like cell clusters may be implanted intraportally into the liver. Alternatively, the pancreatic islet like cell clusters may be implanted into a prevascularized subcutaneous site. Said clusters may be macroencapsulated with a bio-compatible material before implantation. The amount of the clusters to be transplanted will be determined by the art based on the titer of the obtained clusters as well as the general conditions, age, sex, and body weight of the patient to be treated.

Further more, the present invention also provides a method for treating a mammalian patient having disorders in nerve function, which comprises a step of transplanting the nerve like cells derived from allogenic ES cells to the patient. In the present invention, “mammalian patient having disorders in nerve function” includes, but not limited to, patients having nerve degeneration disease such as Alzheimer's disease and Creutzfeldt-Jakob disease or spinal injury. The mammalian patient may include a human patient.

The present invention will be further illustrated by the following Examples. The examples are intended to illustrate but not in any means to limit the invention.

### EXAMPLE 1

Differentiation of Pancreatic Islet Like Clusters from Mouse ES cells

Step 1

Expansion of Undifferentiated ES Cells

In this example, mouse ES cell line 129sv (passages 11; Dainippon Pharmaceutical Co. Ltd., Osaka Japan) was used. A similar study was carried out with mouse ES cell line derived from C57/BL6 mouse (passage 11; kindly provided by Professor Norio NAKATSUJI of Institute for frontier medical sciences, Kyoto University, Kyoto Japan) and similar results as below were obtained (data not shown).

Mammalian ES cells can be proliferated in an undifferentiated state if they are cultured on a feeder layer in the presence of leukemia inhibitory factor. Mouse embryo feeder cells (Dainippon Pharmaceutical Co. Ltd., Osaka, Japan) which had been mitotically inactivated with 20 μg/ml mitomycin were used. ES cell culture medium of high glucose Dulbecco's modified Eagle's medium (D-MEM Cat# 12100: Life Technology, Grand, N.Y.) supplemented with 20% fetal bovine serum replacement (Life Tech.), 2% nonessential amino acid (Life Tech.), 0.1 mmol/l 2-mercaptoethanol (Life Tech.), 1000 U/ml of leukemia inhibitory factor (LIF; Life Tech.) and 2 mmol/l L-glutamine (Life Tech.) was used.

A feeder layer of the mitomycin treated mouse embryonic fibroblasts was prepared on a gelatin-coated culture dish (6 cm), 5 ml of the medium was added thereto and 10⁶ of ES cells were plated on the layer. Cells were cultured at 37° C. in humidified atmosphere of 5% CO₂ in 95% air. Every 3 days, the cells were removed from the dish by means of 0.05% trypsin solution in 0.04% EDTA (Life Tech.) and passaged into a freshly prepared medium on a freshly prepared feeder layer. The ES cells were cultured for 3-7 days.

Step 2

Formation of Embryoid Bodies (EBs)

The ES cells were disassociated by means of the trypsin-EDTA solution and were plated on a non-adherent culture dish to give cell density of 6×10⁵ cells/cm². The cells were kept in suspension culture in the medium used in the above step 1 in the absence or presence of bFGF (4 ng/ml; R&D Systems, Minneapolis, U.S.A. and Kaken Pharmaceuticals, Co. Ltd., Tokyo Japan). Cells were cultured at 37° C. in humidified atmosphere of 5% CO₂ in 95% air. Every 2 days, the media were replaced with freshly prepared ones.

The cultures were daily observed microscopically. At day 2, the cells cultured with bFGF started to aggregate to generate EBs. The suspension culture was kept for 5 days. At day 5, significantly larger number of cell clusters, i.e. EBs were observed in the culture with bFGF than those previously obtained by the conventional EB inducing process without LIF and bFGF (data not shown). In the group without bFGF, only a few aggregation was observed.

Step 3

Selection-Expanding of EBs

The EBs obtained in step 2 with the bFGF containing medium were plated on a Type IV collagen (Sigma, St. Louis, Mo.) coated 6 cm dish filled with the medium used in step 2. After cultured for 48 hr, the medium was replaced with selection-expanding medium of serum free DMEM/F-12(1:1) medium (cat# 11320, Life Tech.) supplemented with 500 μg/ml of Bovine Insulin, 1 μg/ml of Progesterone, 1600 μg/ml of Putrescine and 5 μg 1 ml of Fibronectin and 10 mM of nicotinamide. The cell clusters were cultured for more than 7 days. During the culture, the medium was replaced with freshly prepared medium every 3 days.

Step 4

Differentiation of the Cells

After 7 days culture with the selection-expanding medium, further differentiation was induced by culturing the cell clusters with serum free DMEM/F-12(1:1) medium (cat# 11320, Life Tech.) supplemented with 500 μg/ml of Bovine Insulin, 1 μg/ml of Progesterone, 1600 μg/ml of Putrescine, 1 μg/ml of Laminin and 10 mM of nicotinamide. The cells were incubated at 37° C. in humidified atmosphere of 5% CO₂ in 95% air for 12 days to give islet like cell clusters of about 100-400 μm in diameter.

RNA Extraction and RT-PCR Analysis

At the end of every step as above, pancreatic relating gene expression on the cells was examined by means of RT-PCR analysis.

Cellular RNA of the cells obtained in each step was isolated using ISOGEN (Nippon Gene; Osaka, Japan) according to the manufacturer's instruction. The cells were homogenized in 0.8 ml of ISOGEN using a Potter homogenizer at 4° C. The homogenate was mixed with 1 ml of chloroform, and RNA in the aqueous phase was precipitated with the same volume of isopropyl alcohol. Synthesis of cDNA was carried out with oligo dT primers (Takara Bio Inc., Shiga, Japan) and Moloney murine leukemia virus (M-MLV) Superscript II reverse transcriptase (Gibco/BRL) following the manufacturer's instructions.

Based on thus obtained cDNAs, expression levels of transcription factor mRNAs were determined by means of PCR method. PCR was carried out using standard protocols with Taq polymerase (Boehringer-Mannheim, Indianapolis, Ind.). Cycling parameters were as follows, denaturation at 94° C. for 1 min, annealing at 52-61° C. for 30-120 seconds (depending on the primer) for 1 min, and elongation at 72° C. for 1 min. The number of cycles varied between 25 and 40, depending on the particular mRNA abundance. The number of cycles and the amount of cDNA were chosen in such a way as to select PCR conditions on the linear portion of the reaction curve avoiding “saturation effects” of PCR. Obtained PCR products were confirmed by sequencing.

Primer sequences (forward and reverse), and the length of the amplified products were as follows:

| | |
|---|---|
| β-actin: | |
| ATGGATGACGATATCGCTG | (SEQ ID NO:1) |
| ATGAGGTAGTCTGTCAGGT | (SEQ ID NO:2) |
| 569 bp | |
| nestin: | |
| GGAGTGTCGCTTAGAGGTGC | (SEQ ID NO:3) |
| TCCAGAAAGCCAAGAGAAGC | (SEQ ID NO:4) |
| 327 bp | |
| insulin-I: | |
| TAGTGACCAGCTATAATCAGAG | (SEQ ID NO:5) |
| ACGCCAAGGTCTGAAGGTCC | (SEQ ID NO:6) |
| 288 bp | |
| insulin-II: | |
| CCCTGCTGGCCCTGCTCTT | (SEQ ID NO:7) |
| AGGTCTGAAGGTCACCTGCT | (SEQ ID NO:8) |
| 212 bp | |
| glucagon: | |
| TCATGACGTTTGGCAAGTT | (SEQ ID NO:9) |
| CAGAGGAGAACCCCAGATCA | (SEQ ID NO:10) |
| 202 bp | |
| Islet Amyloid Polypeptide (IAPP): | |
| GATTCCCTATTTGGATCCCC | (SEQ ID NO:11) |
| CTCTCTGTGGCACTGAACCA | (SEQ ID NO:12) |
| 221 bp | |
| Glucose transporter 2 (Glut2): | |
| CCACCCAGTTTACAAGCTC | (SEQ ID NO:13) |
| TGTAGGCAGTACGGGTCCTC | (SEQ ID NO:14) |
| 325 bp | |
| PDX-1: | |
| TGTAGGCAGTACGGGTCCTC | (SEQ ID NO:15) |
| CCACCCCAGTTTACAAGCTC | (SEQ ID NO:16) |
| 325 bp | |
| amylase-2A | |
| CATTGTTGCACCTTGTCACC | (SEQ ID NO:17) |
| TTCTGCTGCTTTCCCTCATT | (SEQ ID NO:18) |
| 300 bp | |
| carboxypeptidase A: | |
| GCAAATGTGTGTTTGATGCC | (SEQ ID NO:19) |
| ATGACCAAACTCTTGGACCG | (SEQ ID NO:20) |
| 521 bp | |
| GATA-4: | |
| CGCCGCCTGTCCGCTTCC | (SEQ ID NO:21) |
| TTGGGCTTCCGTTTTCTGGTTTGA | (SEQ ID NO:22) |
| 193 bp | |
| HNF3: | |
| ACCTGAGTCCGAGTCTGACC | (SEQ ID NO:23) |
| GGCACCTTGAGAAAGCAGTC | (SEQ ID NO:24) |
| 345 bp | |
| OCT-4: | |
| GGCGTTCTCTTTGGAAAGGTGTTC | (SEQ ID NO:25) |
| CTCGAACCAGATCCTTCTCT | (SEQ ID NO:26) |
| 293bp | |

Results are Shown in Table 1 below;

TABLE 1
Gene expression on the cells cultured in the
presence of bFGF in step 2

| | Step 1 | Step 2 | Step 3 | Step 4 |
|---|---|---|---|---|
| OCT4 | + | − | − | − |
| HNF-3β | + | + | + | + |
| Nestin | ± | + | ± | ± |
| Insulin-I | + | + | + | + |
| Insulin-Il | + | + | + | + |
| IAPP | − | ± | ± | + |
| GATA4 | + | + | + | + |
| PDX-1 | + | ± | ± | + |
| Amylase | − | − | − | + |
| carboxypeptidase | − | − | − | + |
| Glut 2 | + | + | + | + |
| Glucagon | − | − | − | + |

The gene of pancreatic transcription factor PDX-1, which is indispensable for pancreatic development, was expressed in steps 1 and 4 cells. Oct-4, which relates to differentiation of ES cell, was expressed in step 1 cells and down-regulated with the differentiation of the ES cells. The ES cells at every step expressed a marker of definitive (embryonic) and visceral (extra-embryonic) endoderm GATA-4 and definitive endoderm HNF3β concerning markers of pancreatic βcell fate. Nestin, a transcription factor relates to immature hormone-negative pancreatic cells, was strongly expressed in step 2 and down-regulated with the differentiation of the ES cells. The results showed that many nestin positive progenitor cells were contained in the EBs obtained in the presence of bFGF. Interestingly, EBs induced by bFGF treatment expressed transcription factors of endocrine (Insulin I, Insulin II, Glucagon, Glucose transporter-2(Glut-2) and Islet Amyloid Polypeptide) specific genes whereas any gene concerning pancreatic islet cells were not expressed in the cell clusters obtained in step 2 using the medium without bFGF. In steps 3 and 4, the cells expressed exocrine specific genes (amylase and carboxypeptidase). These results indicates that pancreatic islet like cell clusters of the invention can be matured to a pancreatic tissue structure, composed of endocrine cells including glucagon-producing a cells, insulin-producing β cells, pancreatic polypeptide-producing γ cells, and somatostatin-producing δ cells and exocrine cells.

Insulin Secretion Test

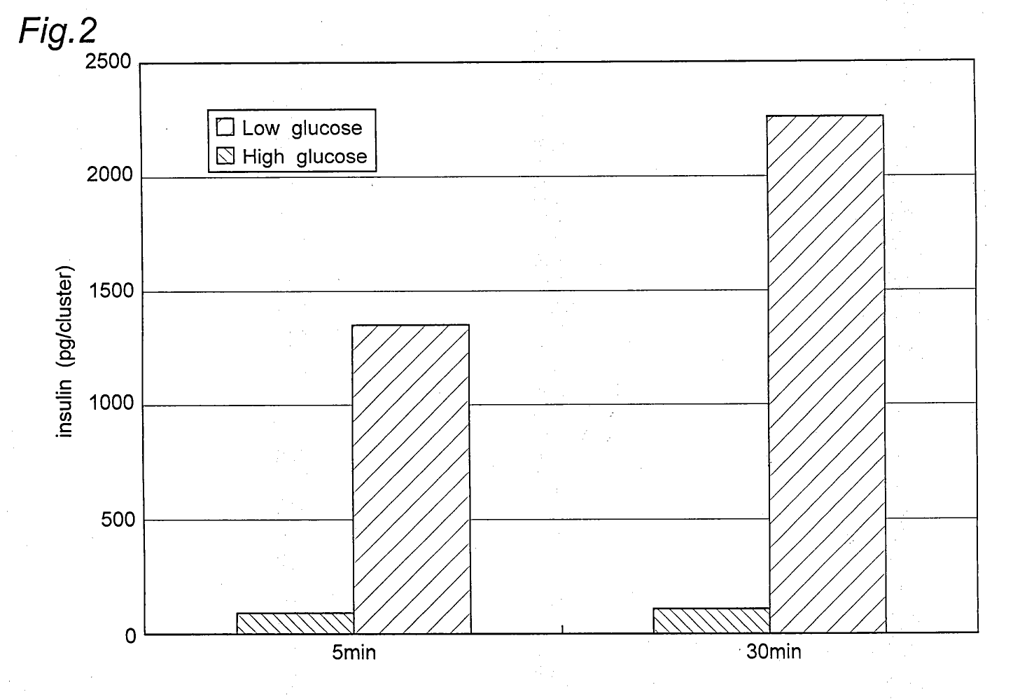

The cell clusters obtained in step 4 (20-25 clusters) were washed 3 times with PBS(−) and plated on a 6 cm cell culture dish containing Krebs-Ringer with bicarbonate buffer consisting of 120 mM NaCl, 5 mM KCl, 2.5 mM CaCl₂, 1.1 mM MgCl₂, 25 mM NaHCO₃ and 0.1% bovine serum albumin, and incubated at 37° C. 3.3 mmol/l (L) or 25 mmol/l (H) glucose was added thereto and incubated. Thirty-five minutes after the glucose stimulation, the insulin contents in the buffer were measured using insulin enzyme-linked immunosorbent assay (ELISA) kit (ALPCO, Windham, N.H.). Results are shown in FIG. 2. In the FIG. 2, the amounts of insulin secreted per one cluster in response to the low or high glucose stimulation at 5 and 30 minutes after the stimulation are shown. The clusters exhibited insulin secretion in response to glucose stimulation in a dose dependent manner.

For determination of total cellular insulin content, the cell clusters obtained in step 4 were extracted with acid ethanol (10% glacial acetic acid in absolute ethanol) overnight at 4° C., followed by sonicating the cells and then, the insulin content in the supernatant was determined by means of the ELISA kit. Total cellular protein amount was determined using DC protein assay system (Bio-Rad laboratories, Hercules, Calif.). The total cellular insulin content of those cell clusters was 71.3 ng/mg protein.

Histological and Immunohistochemical Analysis

Paraffin slices of the cell clusters obtained in step 4 were prepared as follows. The cell clusters (in step 4, incubated 12 days) were washed three times with ice-cold PBS and were fixed with methanol/acetone (1:1) for over night. The clusters were dehydrated with aqueous alcohol (70-100%), then embedded in a paraffin block and the block was sliced to give 4 μm and 8 μm thick slices.

Thus obtained 4 μm thick slices were histologically evaluated with hematoxylin/eosin staining.

In order for Immunohistochemical evaluation, 8 μm thick slices were stained with antibodies by means of the standard protocol. Primary antibodies used herein were as follows:

nestin rabbit polyclonal 1:500 (Dako, Carpinteria, Calif.), tubulin type III (TuJ1) mouse monoclonal 1:500 (Babco, Richmond, Calif.), tubulin type III (TUJ1) rabbit polyclonal 1:2000 (Babco, Richmond, Calif.), insulin mouse monoclonal 1:1000 (Sigma, St. Louis, Mo.), insulin guinea pig polyclonal 1:100 (DAKO, Carpinteria, Calif.), glucagon rabbit polyclonal (DAKO, Carpinteria, Calif.).

In order to detect the primary antibodies, fluorescently labeled secondary antibodies (Jackson Immunoresearch Laboratories, West Grove, Pa.) were used according to the supplier's instruction.

The obtained insulin producing cell cluster was strongly positive to insulin and glucagon, and positive to nestin and TuJ1.

### EXAMPLE 2

Transplantation of the Insulin Producing Cell Clusters into STZ Derived Diabetic Mice.

The insulin producing pancreatic islet like cell clusters were transplanted to determine if the cluster could differentiate into functioning pancreatic islet in vivo.

All animal studies were carried out in accordance with Guideline for Animal Experiments of Kyoto University. Experimental diabetic mice were prepared according to the method disclosed in H. Iwata et al., Pancreas vol. 23(4) 375-381(2001), the disclosure of the publication is herein incorporated by reference. Streptozotocin (STZ) cryopreserved powder (Sigma, St. Louis, Mo.) was dissolved in 0.1 M citrate buffer, pH 4.5 before use. The STZ solution was intraperitoneally injected (227 mg/kg of body weight) to 8- to 10-weeks-old male Nude mice (Shimizu, Kyoto, Japan), Stable hyperglycemia, i.e. increased blood glucose levels of about 350-600 mg/dl) were usually developed 7 to 10 days after the STZ single injection.

Blood glucose level of the mouse was determined using Glucometer Elite XL blood glucose meter (Fujii Corp., Tokyo, Japan). Animals represent 350 mg/dl or more non-fasting blood glucose at 7-10 days of STZ injection were regarded as diabetic mice and used at 14 days from the STZ injection.

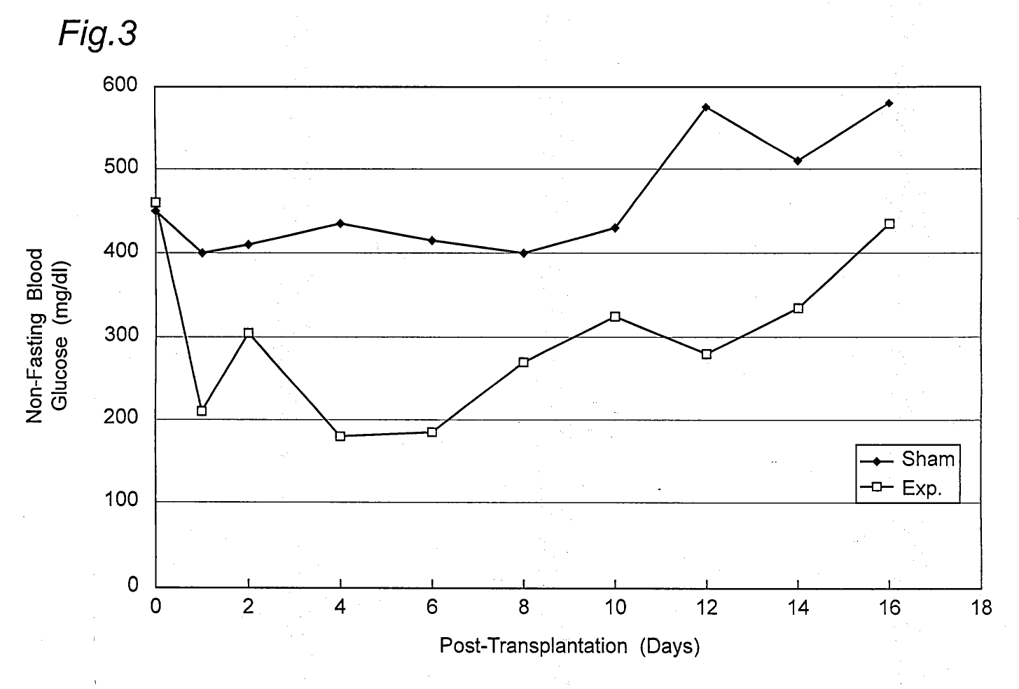

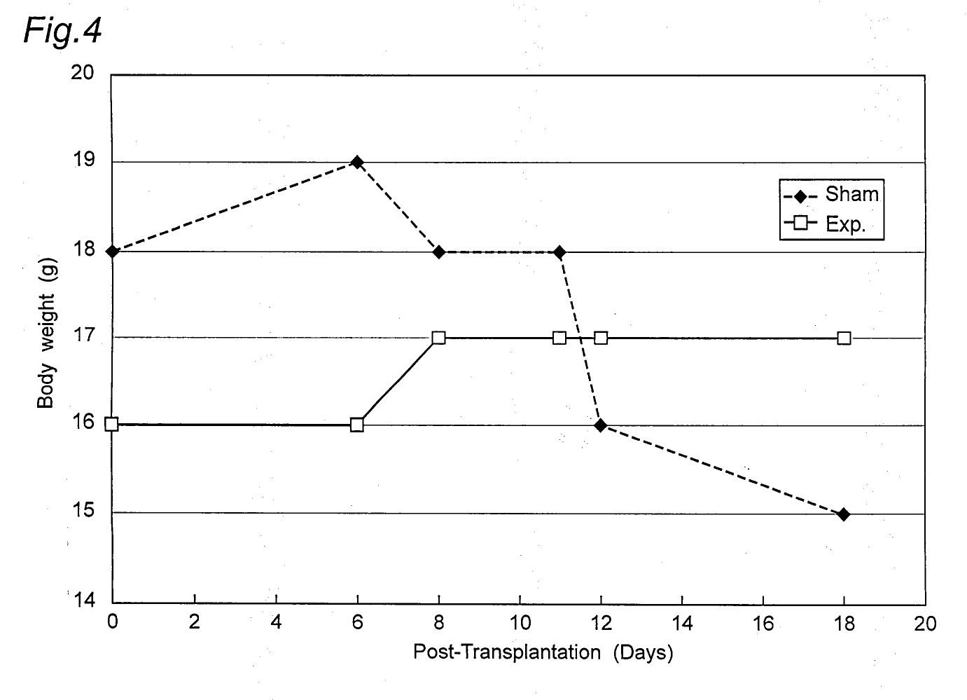

14 days after the STZ injection, the diabetic animals were grafted with 3000 insulin producing pancreatic islet like cell clusters obtained in Example 1 or received sham operation. Under nembutal anesthetization, the cell clusters suspended in PBS(−) were injected into the kidney subcapsular region (one kidney) of the diabetic mice with 23-gauge winged needle. For the sham-operating group, the same volume of PBS(−) was injected in the same manner as above. The experimental group received cell clusters, non-treated control group and sham group consisting of 6, 3 and 3 animals respectively. After the transplantation, non-fasting blood glucose and body weight were monitored daily. The results are shown in FIGS. 3 and 4.

One day after the transplantation, the blood glucose level of the experimental group significantly decreased and remained significantly lower than the sham group throughout the time of the experiment. The body weight of the implanted group increased slightly and remained stable.

At days 14 and 21, 2 and 4 animals implanted with the clusters were sacrificed respectively. All implanted mice remained healthy until killed and kept significantly lower blood glucose than the sham group. To the contrarily, blood glucose levels in non-treated control and sham groups increased gradually and became exhausted. All of the mice of the control and sham groups died prematurely from complications from diabetics between day 14 and day 30 of the operation.

From the sacrificed animals, the implanted tissue was excised, fixed with 4% paraformaldehyde in PBS and embedded in paraffin block. Thus obtained tissue slices of 4-8 μm thickness were immunohistochemically examined in the same manner as Example 1.

At the implanted region, single massed endocrine cells which were immunohistochemically positive for insulin and glucagon were observed. There was no teratoma observed at the area.

### EXAMPLE 3

Induction of Nerve Like Cells

The mouse ES cells, the same as used in Example 1, were treated in the same manner as steps 1-3 of Example 1. Thus obtained cell clusters were then cultured in a dish coated with poly-L-lysine and filled with serum free DMEM/F-12(1:1) medium (cat# 11320, Life Tech.) supplemented with 500 μg/ml of Bovine Insulin, 1 μg/ml of Progesterone, 1600 μg/ml of Putrescine, 10 mM of lysine and 1 μg/ml of Laminin. The cells were cultured for 12 days and the obtained cells were examined genetically and immunohistochemically according to the same manner as described in Example 1. In this example, tyrosine hydroxylase(TH) polyclonal 1:200 (Pel-Freeze, Rogers, Ark.), tyrosine hydroxylase(TH) monoclonal 1:1000 (Sigma, St. Louis, Mo.), serotonin polyclonal 1:4000 (Sigma, St. Louis, Mo.), MAP 2 polyclonal (Chemicon International, Temecula, Calif.), and GFAP monoclonal (Clon Tech, Palo Alto, Calif.) were used in addition to the antibodies used in Example 1.

The obtained cells represented nerve fiber like appearance and were immunohistochemically positive to nestin, TuJ1 (β-tublin III), serotonin, GFAP, MAP 2 and tyrosine-hydroxylase.

By means of RT-PCR described in Example 1, the nerve like cells was confirmed to express Nurt-1 transcription factor. The primer sequences used herein for detecting Nurt-1 were as follows:

| | |
|---|---|
| TGAAGAGAGC GGAGAAGGAG ATC | (SEQ ID NO:27) |
| TCTGGAGTTA AGAAATCGGA GCTG | (SEQ ID NO:28) |
| 255 bp. | |

Accordingly, the obtained nerve like cells are capable of generating mature neurons if they are implanted in vivo.

### EXAMPLE 4

Human ES cells

According to the same manner as described in Example 1, insulin producing pancreatic islet like cell clusters are obtained from human ES cells. ES cells may be those described in the art such as U.S. Pat. Nos. 5,843,780 and 6,200,806; Science 282, 1145-1147 (1998), the disclosures of the publications are herein incorporated by reference. Thus obtained cell clusters produce insulin and secret insulin in response to glucose in a dose dependent manner. The cell clusters are implanted into a human type I diabetic patient. About 3-5 10⁵ clusters are suspended in about 50-100 ml of Krebs-Ringer solution and the suspension is injected into the liver via portal vein, or is implanted to subcutaneous space as a bio-artificial pancreas. The pancreatic function of the implanted patient is restored and the patient acquires independence from insulin.

### EXAMPLE 5

Induction of Dopaminergic (DA) Neuron from Mouse Embryonic Stem Cells

Basically in the same manner as Example 1, differentiation of Mouse ES cell line 129sv (Dainippon-pharm. Co. LTD., Osaka, Japan) was induced.

Step 1

The cells were propagated on a feeder layer of mitomycin treated mouse embryonic fibroblasts in DMEM (Invitrogen, Grand Island, N.Y.) medium containing 20% fetal bovine serum (FBS, JRH Bioscience, Lenexa, Kans.), 1000 unit/ml leukemia inhibitory factor (LIF), 5 mm 2-mercaptoethanol, umm L-glutamic acid and 10× non-essential amino acid (Invitrogen, Grand Island, N.Y.). ES cell colonies were detached and subcultured until a desired amount of the cells is obtained.

Step 2

To induce EB formation, single-cells disassociated by trypsin-EDTA were plated onto nonadherent bacterial dishes at a density less than 6×10⁵ cells/cm² and EBs were kept in suspension culture for 3 days in the medium described above in the presence of bFGF (4 ng/ml; R&D Systems, Minneapolis).

Step 3

After the EBs were formed, they were plated onto Poly (L-lysine)-coated dishes (Falcon Labware, Bedford, Mass.) in the presence of EB culture medium used in the above step. After culturing for 24 hr, the EB culture medium was replaced with serum free DMEM/F-12 (Invitrogen, Grand Island, N.Y.) medium containing 500 μg/ml of bovine insulin, 1 μg/ml of progesterone, 1600 μg/ml of putrescine, 0, 5 or 10 mM of nicotinamide and 5 μg/ml of fibronectin (Nakalai tesque, Kyoto, Japan), and cultured for 4 days.

Step 4

After 4 days of culture, cell differentiation was induced by replacing the medium with serum free DMEM/F-12 medium containing 500 μg/ml of bovine insulin, 1 μg/ml of progesterone, 1600 μg/ml of putrescine, 0, 5 or 10 mM of nicotinamide, 5 μg/ml of fibronectin (Nakalai tesque, Kyoto, Japan) and 1 μ/ml of laminin (Nakalai tesque, Kyoto, Japan). The cells were cultured and then examined as follows.

TuJ-Positive Colonies

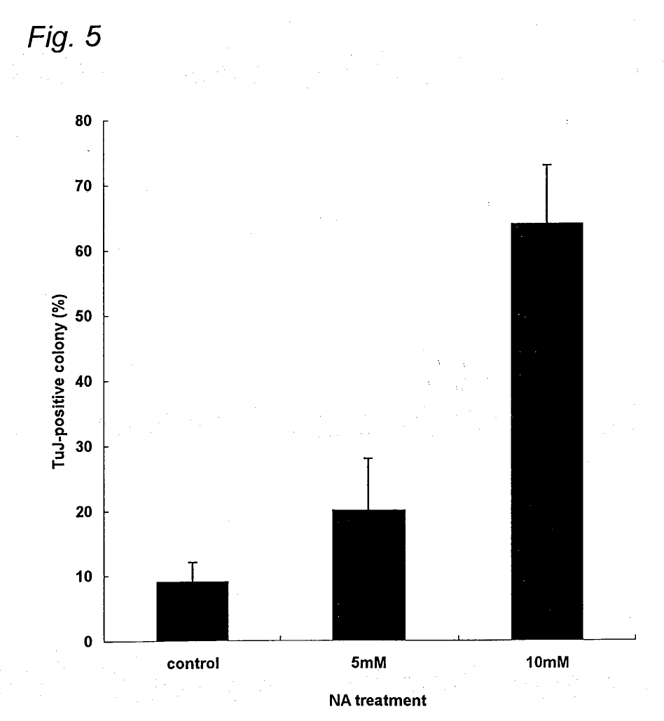

Colonies obtained as above were subjected to Immunohistochemical analysis for expression of the neuronal marker TuJ. Results are shown in FIG. 5.

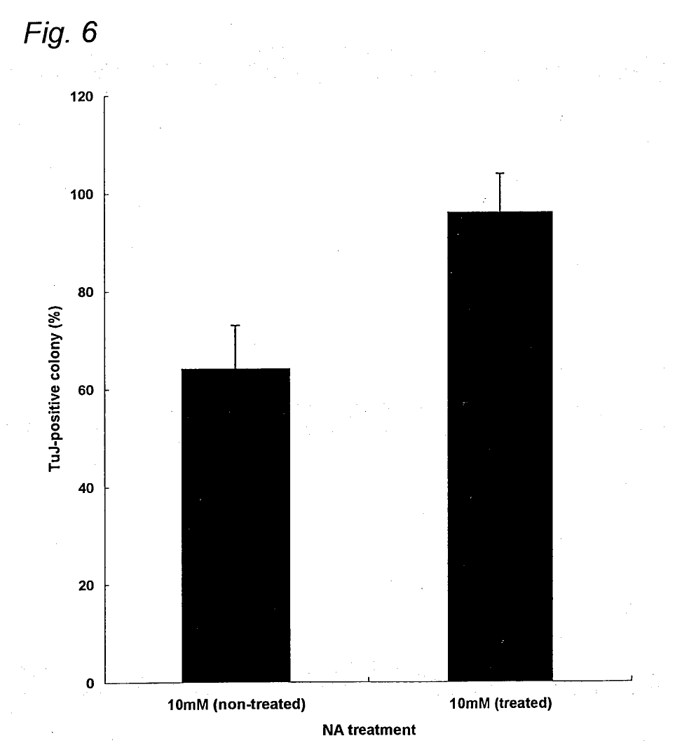

In order to examine the effect of bFGF in step 2, TuJ-expression on the colonies obtained from the ES cells which were treated as above except for cultured in the presence (4 ng/ml) or absence of bFGF in step 2, and then cultured in the presence of 10 mM of nicotinamide in steps 3 and 4 were examined. Results are shown in FIG. 6. In FIG. 6, those cultured in the presence or absence of bFGF are indicated as “treated” or “untreated” respectively.

These results demonstrate that both NA and bFGF enhance the number of colonies that express TuJ.

Expression of Neural Relating Gene on the Cells

By means of RT-PCR described in Example 1, the undifferentiated ES cells (step 1) and those obtained in step 4 were genetically examined. The examined cells were those treated with bFGF in step 2 and cultured in the presence of 10 mM NA in steps 3 and 4. The primers used in this examination are follows:

Nurt-1: tyrosine hydroxylase

| | |
|---|---|
| TGAAGAGAGC GGAGAAGGAG ATC | (SEQ ID NO:27) |
| TCTGGAGTTA AGAAATCGGA GCTG | (SEQ ID NO:28) |

Wnt-1: Mesencephalic dopaminergic neuron
transcription factor
signaling molecule

| | |
|---|---|
| 5′-ACCTGTTGACGGATTCCAAG-3′ | (SEQ ID NO:29) |
| 5′-TCATGAGGAAGCGTAGGTCC-3′; | (SEQ ID NO:30) |

Engrailed-1 (En 1): Mesencephalic dopaminergic
neuron transcription factor

| | |
|---|---|
| 5′-TCAAGACTGACTCACAGCAACCCC-3′ | (SEQ ID NO:31) |
| 5′-CTTTGTCCTGAACCGTGGTGGTAG-3′ | (SEQ ID NO:32) |

Ptc: SHH signal receptor, patched

| | |
|---|---|
| 5′-CCTCCTTTACGGTGGACAAA-3′ | (SEQ ID NO:33) |
| 5′-ATCAACTCCTCCTGCCAATG-3′ | (SEQ ID NO:34) |

Glycoprotein sonic hedgehog (Shh)

| | |
|---|---|
| 5′-GGAAGATCACAAGAAACTCCGAAC-3′ | (SEQ ID NO:35) |
| 5′-GGATGCGAGCTTTGGATTCATAG-3′ | (SEQ ID NO:36) |

Otx 1: Transcription factor critical in brain
morphogenesis

| | |
|---|---|
| 5′-GCTGTTCGCAAAGACTCGCTAC-3′ | (SEQ ID NO:37) |
| 5′-CCATGACCTATACTCAGGCTTCAGG-3′; | (SEQ ID NO:38) |

Otx 2: Transcription factor critical in brain
morphogenesis

| | |
|---|---|
| 5′-CCATGACCTATACTCAGGCTTCAGG-3′ | (SEQ ID NO:39) |
| 5′-GAAGCTCCATATCCCTGGGTGGAAAG-3′ | (SEQ ID NO:40) |

Pax 2: Mesencephalic dopaminergic neuron
transcription factor

| | |
|---|---|
| 5′-CCAAAGTGGTGGACAAGATTGCC-3′ | (SEQ ID NO:41) |
| 5′-GGGATAGGAAGGACGCTCAAAGAC-3′ | (SEQ ID NO:42) |

Pax 5: Mesencephalic dopaminergic neuron
transcription factor

| | |
|---|---|
| 5′-CAGATGTAGTCCGCCAAAGGATAG-3′ | (SEQ ID NO:43) |
| 5′-ATGCCACTGATGGAGTATGAGGAGCC-3′ | (SEQ ID NO:44) |

Smo: SHH signal receptor, smoothened

| | |
|---|---|
| 5′-CTGAGAGTGCCAGAAAAGGG-3′ | (SEQ ID NO:45) |
| 5′-TCATCATGCTGGAGAACTCG-3′ | (SEQ ID NO:46) |

Fibroblast growth factor receptor (FGF 3R)

| | |
|---|---|
| 5′-ATCCTCGGGAGATGACGAAGAC-3′ | (SEQ ID NO:47) |
| 5′-GGATGCTGCCAAACTTTGTTCTC-3′ | (SEQ ID NO:48) |

Results are Shown in Table 2

TABLE 2

| | Nurt-1 | Nurt-1 | Wnt-1 | En-1 | Ptc | Shh |
|---|---|---|---|---|---|---|
| Step 1 | − | + + + | − | − | + | − |
| Step 4 | + + | + + + | + + + | + + + + | + + + | + + + |
| | Otx-1 | Otx-2 | Pax-2 | Pax-5 | SMO | FGF-3R |
| Step 1 | − | + + | − | − | + | − |
| Step 4 | + + | + + + + | + + + + | + + + + | + | + + |

As is shown in table 2, colonies induced according to the instant invention expressed high level of neural specific genes encoding transcription factors and signaling molecules.

Development of Neural Network Like Appearance

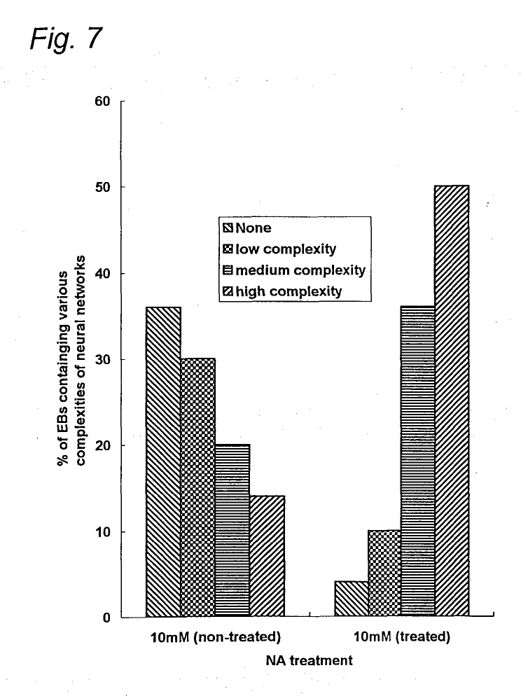

The obtained colonies were microscopically examined to determine the degree of complexities of the neural network like appearance. Results are shown in FIG. 7. FIG. 7 is histogram summarizing the effect of NA (nicotinamide) and bFGF on complexities of the neural network like appearance of the EBs. The EBs were cultured in the presence or absence of 4 ng/ml of bFGF in step 2 (indicated “non-treated” or “treated” respectively) and then cultured in the presence of NA in steps 3 and 4. Each condition was assayed by counting all EBs in the culture and the experiment was repeated twice resulting in similar results.

BMP-4 or FBS Treatment

The cells treated with bFGF in step 2 were then cultured in the presence of 0.5 nM bone morphogenetic protein (BMP-4) or 5% fetal bovine serum (FBS) in addition to 10 mM of NA in the following steps (steps 3 and 4). In this procedure, BMP4 was freshly added at each medium change. Both BMP-4 and FBS suppress differentiation of the cells into neural cells.

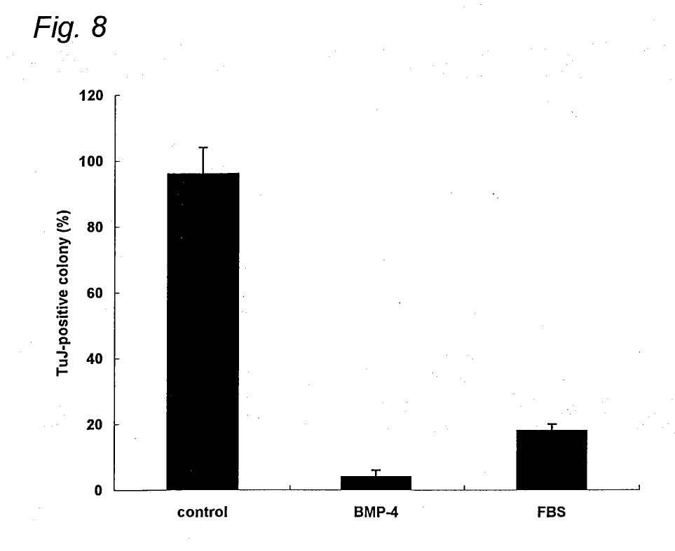

Results are shown in FIG. 8. Both of BMP-4 and FBS suppressed the expression of TuJ on the colonies.

Effects of Transplantation of the Differentiated Cells on 6-OHDA Lesion and Rotational Behavior

It is considered that a unilateral dopaminergic neurogenerative animal induced by 6-OHDA is a useful model of Parkinson's disease (PD), since loss of nigral dopaminergic neurons is characterized in PD pathology. It is known that marked dopamine depletion (over 95%) in the striatum induces the supersensitivity in dopamine receptors. In 6-OHDA-lesioned rats, therefore, a mixed D1/D2 receptor agonist apomorphine induces rotational asymmetry in the contralateral direction, while a DA releaser amphetamine induces the ipsilateral rotation. Accordingly, the inventors studied in vivo effects of transplantation of the differentiated and undifferentiated ES cells on the 6-OHDA lesion model.

All animal studies were in accordance with Kyoto University guidelines and the National Institutes of Health Guide for the Care and Use of Laboratory Animals. Experimental animal used were 8-week-old male Wister rats, weighing approximately 280 g (SLC Inc., Hamamatsu, Japan). The rats were fasted overnight with free access to water. 6-Hydroxydopamine (6-OHDA) solution was prepared by dissolving 12 g of 6-OHDA (Sigma, St Louis, Mo.) in 4l of sterilized physiological saline containing 0.02% ascorbic acid. For stereotaxic microinjection, rats were anesthetized (sodium pentobarbital, 50 mg/kg, i.p.) and immobilized in Kopf stereotaxic frame. Subsequently, the rats were injected with the 6-OHDA solution into left substantia nigra, where the bregma was −4.8 mm caudal, 1.8 mm left lateral, −7.8 mm ventral, via a motor-driven 10 μl-Hamilton syringe using 26 gauge needle. Coordinates were set according to the rat brain atlas (Paxinos G. and Watson C. (1986) “The rat brain in stereotaxic coordinates” (Second Edition). Academic Press, North Ryde, Australia, the contents of which are hereby incorporated by reference). After 2 weeks, rats for transplantation were selected from the lesioned rats based on their rotational behavior in response to apomorphine (Sigma). The rotational test was performed in the rotometer bowls (Ungerstedt, U. (1971) “Postsynaptic supersensitivity after 6-hydroxydopamine induced degeneration of the nigro-striatal dopamine system” Acta Physiol. Scand., 82 Suppl. 367, 69-93, the contents of which are hereby incorporated by reference) and the total number of full 360° rotations in contralateral directions was counted. Animals that showed a strong contralateral rotational behavior in response to apomorphine (0.6 mg/kg, i.p.) were selected for the following transplantation surgery.

Transplantation Surgery

6-OHDA-lesioned rats were injected with the vehicle (sterilized phosphate-buffered saline (PBS) n=10, a: vehicle) or cell suspension containing low numbers of undifferentiated ES cells (about 4000 cells of ES cells from step 1, n=10, b: ES-L), and differentiated cells (about 20,000 cells of the cells from step 4 day 4, n=10, i: 4F).

The selected rats were anesthetized (sodium pentobarbital, 50 mg/kg, i.p.) and then placed in Kopf stereotaxic frame. Each animal received a microinjection of 1.0 μl of the grafting cell suspension or the vehicle into two sites of the left striatum, where the bregma was +1.0 mm caudal, 3.0 mm left later, −5.5 and 5.0 mm ventral according to the rat brain atlas (id), using a 10 μl-Hamilton syringe with 22 gauge needle. A 5-min waiting period allowed transplanted cells to settle before the needle was removed. Subsequently, apomorphine-induced rotational asymmetry was assessed every 2 weeks.

The number of full 360° rotations in the contralateral direction was counted for 60 min after administration of apomorphine (0.6 mg/kg, i.p.).

Statistics

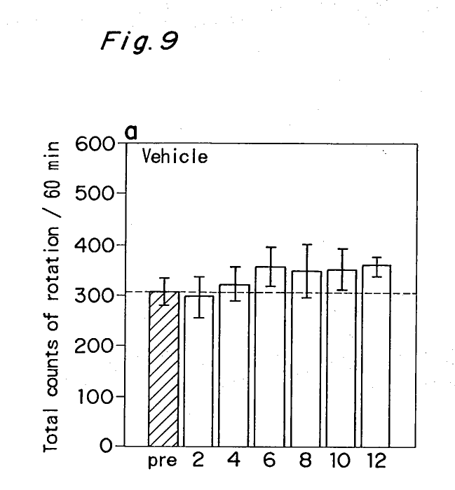

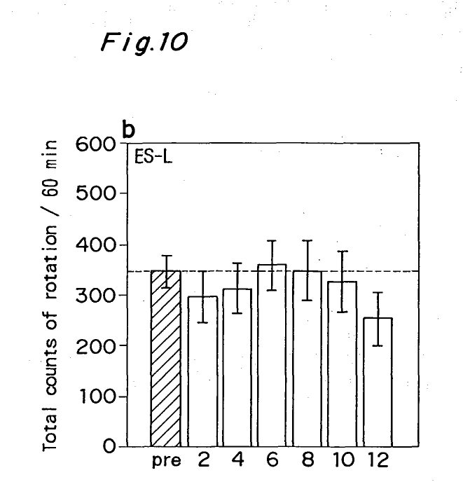

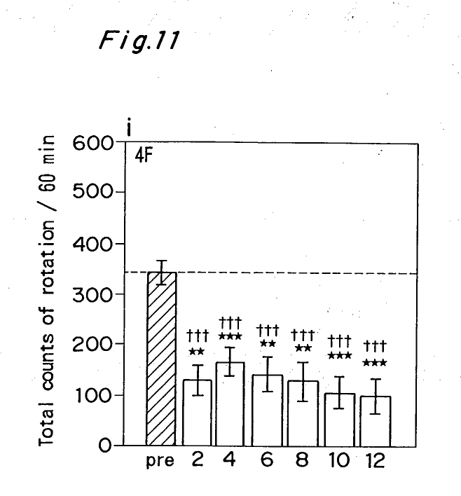

Results are shown in FIGS. 9-11. Data are presented as mean±SEM and compared using the unpaired Student's test or one-way analysis of variance. Differences were considered significant at P<0.05. Further statistical analysis for post hoc comparison was performed using the Bonferroni/Dunn test (Statview, Abacus Concepts, Berkely, USA) for the numbers of apomorphine-induced rotational asymmetry.

Apomorphine-induced rotation asymmetry was significantly improved by transplantation of differentiated DA-neurons in step 4 after 2 weeks. However, other transplanted cells did not efficiently contralateral rotation. Significance (Bonferroni/Dunn post hoc comparisons after ANOVA): *p<0.05, **p<0.01, ***p<0.001 vs. each time point in the vehicle-injection (a); ! p<0.05, !!! p<0.001 vs. pre-transplantation in each group.

In this study, undifferentiated mES cells and the vehicle did not improve apomorphine induced rotational asymmetry. In contrast, transplantation of the ES cells treated according to the instant invention (at step 4) significantly reduced the apomorphine induced rotational asymmetry after 2 weeks. This restoration sustained until the end of behavioral assessment at 12 weeks.

According to the results of the example 5, it can be concluded that mouse ES cells were differentiated into dopaminergic nerve cells according to the method of the present invention.

According to the same manner as described in the above examples, nerve like cell can be obtained from human ES cells. Human ES cells may be those described in Example 4. Although some minor modifications would be required for optimizing the culture conditions, it would be mere routine work for the art. According to the present invention, dopaminergic neurons can effectively enriched from the total population of differentiated ES cells without gene modification, flow cytometry sorting or separation with magnetic beads. The nerve like cells induced from ES cells by the simple 4-step method of the instant invention represent good therapeutic effect on Parkinson's disease and the patients receiving the treatment can survive long-term.

What is claimed is:

1. A method for inducing differentiation of mammalian embryonic stem cells into functioning cells, which comprises the steps of:
1) culturing the mammalian embryonic stem cells together with feeder cells with a medium comprising leukemia inhibitory factor;
2) culturing the cells obtained from step 1) in absence of feeder cells with a medium comprising leukemia Inhibitory factor and basic FGF in a suspension culture condition to give embryonic bodies;
3) culturing the obtained embryonic bodies with a selection-expanding medium; and
4) culturing the obtained cell clusters with a differentiation medium to give functioning cells.

2. The method of claim 1, wherein the medium used in step 2) comprises about 100-10000 U/ml of leukemia inhibitory factor.

3. The method of claim 1, wherein the medium used in step 2) comprises about 2-100 ng/ml of bFGF.

4. The method of claim 1, wherein the medium used in step 3) comprises nicotinamide, insulin and fibronectine in a serum-free cell culture medium.

5. The method of claim 1, wherein the functioning cells are insulin producing pancreatic islet-like cell clusters.

6. The method of claim 5 wherein the medium used in step 4) comprises nicotinamide, insulin and laminin in a serum-free cell culture medium.

7. The method of claim 1, wherein the functioning cells are nerve-like cells.

8. The method of claim 7 wherein the medium used in step 4) comprises L-lysine, insulin and laminin in a serum-free cell culture medium.

9. Functioning cells induced from mammalian ES cells by the method of claim 1.

10. Insulin secreting cell clusters induced from mammalian ES cells by the method of claim 5.

11. Nerve-like cells induced from mammalian ES cells by the method of claim 7.

12. A method for treating a mammalian patient having disorders in pancreatic islet function, which comprises implanting pancreatic islet-like cell clusters induced from allogenic ES cells by the method of claim 5 to the patient.

13. The method of claim 12, wherein the patient is a type I diabetic patient.

14. A method for treating a mammalian patient having disorders in nerve function, which comprises implanting nerve-like cells induced from allogenic ES cells by the method of claim 7 to the patient.
